(19) United States
(12) Patent Application Publication
(10) Pub. No.: US 2010/0143406 A1
(43) Pub. Date: Jun. 10, 2010

(54) METHODS OF ENHANCING PROTEIN INCORPORATION INTO VIRUS LIKE PARTICLES

(76) Inventors: Gale SMITH, Rockville, MD (US); Peter Pushko, Rockville, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/306,965

(22) PCT Filed: Jun. 27, 2007
(86) PCT No.: PCT/US07/72272

§ 371 Date: Feb. 1, 2010

Related U.S. Application Data
(60) Provisional application No. 60/817,402, filed on Jun. 30, 2006.

(51) Int. Cl. A61K 39/12; C12N 7/00

(52) U.S. Cl. 424/204.1; 435/235.1

(57) ABSTRACT

The present invention comprises a method of increasing glycoprotein incorporation on the surface of VLPs, comprising expressing a nucleic acid encoding a chimeric glycoprotein in a host cell, wherein said chimeric glycoprotein comprises the transmembrane domain of an influenza hemagglutinin protein. The invention also embodies specific VLPs comprising said chimeric glycoproteins and methods of inducing immunity in an animal utilizing said VLPs.

This application claims priority to provisional application 60/817,402, filed Jun. 30, 2006, which is herein incorporated by reference in its entirety for all proposes.

### ACKNOWLEDGMENT OF GOVERNMENT SUPPORT

The research that led to this invention was partially funded by Government support under Grant number 5 U19AI28147-18 awarded by the National Institutes of Health.

### BACKGROUND OF THE INVENTION

Virus-like Particles (VLPs) assemble spontaneously with the expression of viral structural proteins in vivo and in cultured cells including insect cells (Noad, R., et al., (2003) Trend in Micro., 11, 438 to 444). Virus-like particles (VLPs) closely resemble mature virions, but they do not contain viral genomic material (i.e., viral genomic RNA). Because of this lack of genomic material, VLPs are nonreplicative in nature, which make them safe for administration in the form of an immunogenic composition (e.g., vaccine) with a broad range of viruses including influenza virus (Pushko, P., et al., (2005) Vaccine, 23, 5751-5759; Galarza, J., et al., (2005) Viral Immunol., 18, 365-372), Hepatitis B (Bohm, W., et al. (1995) J. Immunol., 155, 3313-3321), Human immunodeficiency virus (Young, K., et al. (2003) Curr. Drug Targets Infect. Disord., 3, 151-159) and human papillomavirus (Harro, C., et al. (2001) J. Natl. Cancer Inst., 93, 284-292). VLPs represent an alternative to inactivated or subunit vaccines with the advantage that structural antigens are presented in a noninfectious form in the absence of a viral genome, while maintaining the integrity of conformationally dependent antigenic epitopes. VLPs may be intrinsically more immunogenic because they can present repetitive protein or carbohydrate arrays that have a potential to activate pathogen-associated molecular pattern (PAMP) recognition toll-like receptors on antigen-presenting calls that would stimulate innate immune response (Lenz P, et al., (2001) J. Immunol., 166, 5346-5355).

Many viruses, like Human Immunodeficiency virus type 1 (HIV-1), influenza, and Ebola have an internal core containing the viral genome and an outer membrane consisting of a lipid bilayer derived from the host cell's plasma or integral membrane and one or more envelope proteins that are embedded in the virus envelope, for example HIV-1 GP160 ENV or influenza Hemaglutinin (HA) glycoproteins. The co-expression of these envelope proteins along with a core, capsid, matrix or tegument proteins can lead to assembly of VLPs. The VLPs that bud from the intracellular membranes, like from the golgi, may or may not secreted from the cell.

Baculoviruses can be genetically modified to express influenza hemaggutinin (HA), neuraminidase (NA) and matrix (M1) proteins (Pushko, P., et al., (2005) Vaccine, 23, 5751-5759). HA, NA and M1 self assemble in Spodoptera frugiperda (Sf9) insect cells into particles resembling mature influenza enveloped nucleocapids. HIV-1 VLPs are made by co-infection with two or a single “tandem” baculovirus vector which produce the major HIV-1 capsid protein p55 Gag and the HIV-1 envelope glycoprotein GP160 Env. The structural proteins from HIV-1 (ENV, GP160 and p55 Gag) are assembled in Sf9 insect cells and secreted as VLPs from about 36 hours to about 72 hours post infection with baculovirus expression vector which contain these genes under the transcriptional control of the polyhedrin or other suitable promoter.

Complex viruses such as HIV-1 and influenza cannot be assembled in vitro from individual components due to the complex nature of viruses with a lipid bilayer envelope. Therefore, the efficiency of incorporation of envelope proteins into VLPs is a function of the intrinsic properties of the structural proteins and lipids required for VLP formation. Assembly of complex VLPs occurs in vivo as part of the budding process of capsid structures from integral or plasma membranes. Given that HIV-1 gp160 is the receptor of the human immunodeficiency virus and that specific antibodies against HIV-1 can neutralize the virus, it would be advantageous to drive and increase HIV viral and/or other glycoproteins to the surface of VLPs.

### SUMMARY OF THE INVENTION

The present invention comprises a method of increasing glycoprotein incorporation on the surface of VLPs, comprising expressing a nucleic acid encoding a chimeric glycoprotein in a host cell with a non-influenza viral core or matrix protein, wherein said chimeric glycoprotein comprises the transmembrane domain of an influenza hemagglutinin protein. In another embodiment, said chimeric glycoprotein further comprises the carboxyl terminal tail of an influenza hemagglutinin protein. In another embodiment, said glycoprotein is derived from a viral glycoprotein.

The invention also comprises a virus like particle comprising at least one chimeric glycoprotein on the surface of the VLP and a non-influenza viral core or matrix protein, wherein said chimeric glycoprotein comprises the transmembrane domain of an influenza hemagglutinin protein. In another embodiment, said chimeric glycoprotein further comprises the carboxyl terminal tail of an influenza hemagglutinin protein

The invention also comprises a vaccine or immunogenic composition comprising a virus like particle comprising at least one chimeric glycoprotein on the surface of the VLP and a non-influenza viral core or matrix protein. In one embodiment, said chimeric glycoprotein comprises the transmembrane domain of an influenza hemagglutinin protein. In another embodiment, said chimeric glycoprotein further comprises the carboxyl terminal tail of an influenza hemagglutinin protein

The invention also comprises a method of inducing protective immunity in an animal, comprising administering to said animal a VLP comprising at least one chimeric glycoprotein on the surface of the VLP and a non-influenza viral core or matrix protein, wherein said chimeric glycoprotein comprises an influenza hemagglutinin transmembrane domain.

### BRIEF DESCRIPTION OF THE DRAWINGS

FIG. 1 depicts the HIV-1 chimeric env genes that were cloned in the baculovirus expression vectors and co-expressed in Sf9 insect cells with either HIV-1 p55 Gag or influenza M1 matrix proteins to form a homotypic or heterotypic VLPs with chimeric HIV-1 gp145 influenza HATM or HATM with HACT modifications.

FIG. 2 depicts SDS page (left panel) or western blots (middle and right panel) of isolated VLPs.

FIG. 3 depicts a western blot of processed VLPs comprising chimeric HIV-Env glycoprotein, including C-TM.CT_{HA}. In the lower panel, Env and Gag contents were quantitated by sandwich ELISA.

### DETAILED DESCRIPTION OF THE INVENTION

As used herein the term “adjuvant” refers to a compound that, when used in combination with a specific immunogen (e.g. a VLP) in a formulation, augments or otherwise alters or modifies the resultant immune response. Modification of the immune response includes intensification or broadening the specificity of either or both antibody and cellular immune responses. Modification of the immune response can also mean decreasing or suppressing certain antigen-specific immune responses.

As use herein, the term “animal” refers to humans and other primates, including non-human primates such as chimpanzees and other apes and monkey species. Farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like are also non-limiting examples. Both adult and newborn individuals are intended to be covered.

As used herein, the term “baculovirus” also known as baculoviridae, refers to a family of enveloped DNA viruses of arthropods, members of which may be used as expression vectors for producing recombinant proteins in cell cultures. The virion contains one or more rod-shaped nucleocapsids containing a molecule of circular supercoiled double-stranded DNA (Mr 54×10⁶-154×10⁶). The virus used as a vector is generally Autographa californica nuclear polyhedrosis virus (NVP). Expression of introduced genes are under the control of the strong promoter that normally regulates expression of the polyhedron protein component of the large nuclear inclusion in which the viruses are embedded in the infected cells.

As used herein, the term “chimeric” or “chimeric glycoprotein” refers a glycoprotein that comprises the amino acid sequence of an influenza hemagglutinin transmembrane domain. Such domain can be in any portion of the glycoprotein and/or can replace the natural transmembrane domain of said glycoprotein. The term also encompasses a glycoprotein which comprises the influenza hemagglutinin C-terminal domain with or/without said influenza transmembrane domain. The term also encompasses a glycoprotein which comprises the influenza hemagglutinin cytoplasmic domain with or/without said influenza transmembrane domain. Also contemplated as an embodiment of the invention are chimeric proteins that are not glycosylated.

As used herein, the term “derived from” refers to the origin or source, and may include naturally occurring, recombinant, chimeric, unpurified, or purified molecules.

As used herein, the term “derivative” in the context of a protein or peptide (e.g., gp160) refers to a protein or peptide that comprises an amino acid sequence that has been altered by the introduction of amino acid residue substitutions, deletions, and/or additions. The term “derivative” as used herein also refers to a protein or peptide which has been modified, i.e., by the covalent attachment of any type of molecule to the protein or peptide. For example, but not by way of limitation, a protein or peptide may be modified, e.g., by glycosylation.

As used herein, the term “vaccine” refers to a suspension or solution of an immunogen (e.g. VLP) that is administered to an animal to produce protective immunity, i.e., immunity that reduces the severity of disease associated with an infection.

As used herein the term “immune response” refers to the induction of the immune system in an animal (e.g., a human). The induction of the immune system refers to the stimulation of a cell-mediated immunological response and/or a humoral response.

As use herein, the teen “antigenic formulation” or “antigenic composition” refers to a preparation which, when administered to an animal, will induce an immune response.

As use herein, the term “purified VLPs” refers to a preparation of VLPs of the invention that is at least 50%, 55% 60%, 65%, 70%, 75%, 80%, 85%, 90%, 95%, 96%, 97%, 98%, 99% or greater, substantially free from other molecules (exclusive of solvent) in a mixture. For example, VLPs of the invention can be substantially free of other viruses, proteins, lipids, and carbohydrates associated with making VLPs of the invention.

The invention discloses a method where glycoproteins comprising a transmembrane domain and/or cytoplasmic domain from influenza hemagglutinin protein is efficiently assembled into VLPs and secreted from Sf9 insect cells. As a specific example, the HIV-1 Env protein (e.g. gp160) comprising the influenza HA transmembrane domain and/or cytoplasmic domain is co-expressed with p55 Gag or M1 influenza to form VLPs in which the chimeric Env proteins are expressed on the surface of said VLPs more efficiently than non-chimeric HIV Env proteins. This invention is useful for improving the production of VLPs and to produce novel VLPs with the potential for use in the prevention and/or treatment of disease or for diagnostic applications. The invention also encompasses a method of producing VLPs from any peptide, polypeptide, protein and/or glycoprotein by fusing said peptide, polypeptide, protein and/or glycoprotein to influenza HA transmembrane domain and/or cytoplasmic domains. These chimeric constructs will be displayed on the surface of VLPs. The increased display of immunogenic glycoproteins on the surface of VLPs is advantageous for increasing the immune response in a mammal.

Method of Increasing Protein Expression on the Surface of VLPs

VLPs of the invention are useful for preparing vaccines and immunogenic compositions. One important feature of VLPs is the ability to express surface glycoproteins so that the immune system of an animal induces an immune response against said glycoprotein. However, not all glycoproteins can be expressed on the surface of VLPs. There may be many reasons why certain glycoproteins are not expressed, or be poorly expressed, on the surface of the VLPs. One reason is that said glycoprotein is not directed to the membrane of a host cell or that said glycoprotein does not have a transmembrane domain. The inventors have discovered that HIV VLPs have a very low level of HIV-1 envelope gp160 that assembles with HIV-1 p55 core, thus reducing the surface expression of gp160, and derivative proteins, on the surface of VLPs. Given that HIV gp160 is the receptor of the virus, and that specific antibodies against HIV-1 can neutralize the virus, low levels of gp160 on the surface of HIV VLPs can severely limit their use as a vaccine. Therefore, the use of VLPs as a vaccine alternative can be reduced due to lack of expression of antigens on the surface of VLPs because there is no mechanism to efficiently drive certain useful glycoproteins to the surface of the VLP.

Sequences near the carboxyl terminus of influenza hemagglutinin may be important for incorporation of HA into the lipid bilayer of the mature influenza enveloped nucleocapsids and for the assembly of HA trimer interaction with the influenza core protein M1 (Ali, et al., 2000). Thus, the invention comprises chimeric glycoproteins that comprise the transmembrane domain of influenza hemagglutinin and/or the c-terminal (CT) region (cytoplasmic region) of the influenza hemagglutinin protein. The invention also comprises methods of making VLPs comprising said chimeric glycoproteins. These chimeric glycoproteins are efficient at being incorporated into the membrane of a host cell, allowing formation of VLPs and being expressed on the surface of VLPs. These disclosed methods takes advantage of the efficient system utilized by influenza virus to make virus particles.

Thus, the invention comprises a virus like particle comprising at least one chimeric glycoprotein on the surface of the VLP. In one embodiment, the VLP comprises the transmembrane domain of an influenza hemagglutinin glycoprotein (HATM). The HATM can be incorporated into the glycoprotein in any section of the protein. In one embodiment, the HATM is fused toward the carboxyl terminal of a glycoprotein. In another embodiment, the HATM is fused toward the amino terminal of a glycoprotein. In another embodiment, the HATM replaces at least one natural transmembrane domain of the native glycoprotein.

In another embodiment, said VLP further comprises the carboxyl terminal tail of an influenza hemagglutinin protein (HACT). The carboxyl terminal tail of the HA is important for associating with the M1 protein of influenza virus. As shown below, the inventors have discovered that a chimeric glycoprotein comprising HATM and/or HATC also associates with other viral cores, e.g. HIV p55 Gag. Thus, a glycoprotein with a HACT will be driven to the plasma membrane of the host cell before said cell buds and releases a VLP. In one embodiment, said HACT will be fused to the carboxyl terminal of a glycoprotein. In another embodiment, the HACT will be fused to the amino terminal end of a glycoprotein. In another embodiment, a VLP will comprise a chimeric protein comprising HACT fused to the amino terminal end of a glycoprotein. In another embodiment, said VLP further comprises a viral core protein (or a homologous protein). In another embodiment, said core protein is an influenza M1 protein. In one embodiment, the core or matrix protein is from a virus other than influenza. In another embodiment, said core protein comprises HIV p55 Gag protein. In another embodiment, said glycoprotein will comprise both the HACT domain and the HATM domain.

Any non-influenza core or matrix protein could be expressed with the chimeric proteins of the invention to make VLPs of the invention. Non-limiting examples of said core or matrix proteins are SIV Gag (Yamshchikov, G., et al. (1995) Virology, 214, 50-58) and parainfluenza M (Coronel E. C., et al., (1999) J. Virol., 73, 7035-7038).

Non-limiting examples of viruses from which said chimeric glycoproteins can be derived are from the following: seasonal, avian or pandemic influenza virus (A and B, e.g. HA and/or NA), coronavirus (e.g. SARS), hepatitis viruses A, B, C, D & E3, human immunodeficiency virus (HIV), herpes viruses 1, 2, 6 & 7, cytomegalovirus, varicella zoster, papilloma virus, Epstein Barr virus, parainfluenza viruses, adenoviruses, bunya viruses (e.g. hanta virus), coxsakie viruses, picoma viruses, rotaviruses, rhinoviruses, rubella virus, mumps virus, measles virus, polio virus (multiple types), adeno virus (multiple types), parainfluenza virus (multiple types), shipping fever virus, Western and Eastern equine encephalomyelitis, Japanese encephalomyelitis, fowl pox, rabies virus, slow brain viruses, rous sarcoma virus, Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), Togaviridae (e.g., Rubivirus), respiratory syncytial virus (RSV), West Nile fever virus, Tick borne encephalitis, yellow fever, chikungunya virus, and dengue virus (all serotypes), Astrovirus, calicivirus (including norovirus), picornaviridae (including enterovirus), arenaviruses, and lymphochoriomeningitis virus.

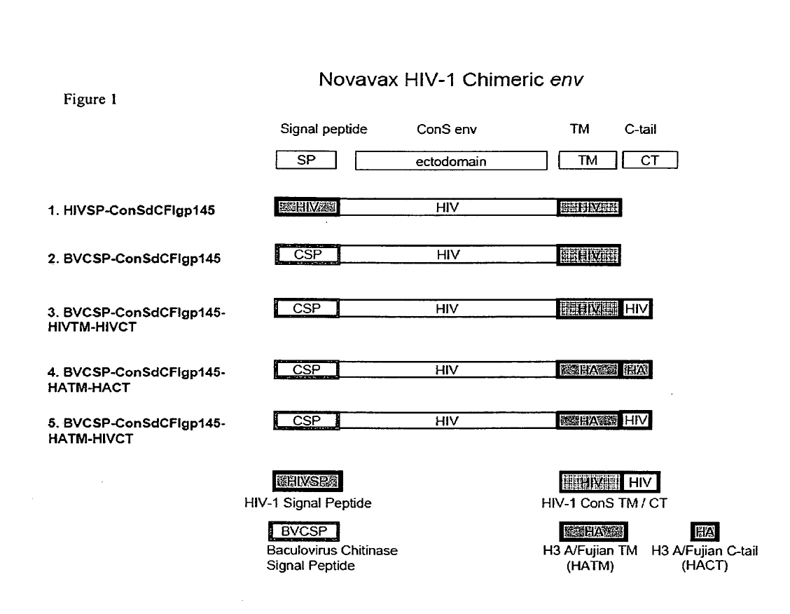

In one embodiment, said viral glycoprotein is a HIV glycoprotein. In another embodiment, said HIV glycoprotein is gp160, gp120, gp41 and/or gp145 or derivatives thereof. In another embodiment, said nucleic acid sequences comprise the sequence that codes for any one of SEQ ID NO 2 to SEQ ID NO 8. In another embodiment, said sequence encodes for a signal sequences derived from the gene of an insect. In another embodiment, said signal sequence is from the chitinase gene. In another embodiment, said VLP comprises the amino acid sequence that codes for any one of SEQ ID NO 2 to SEQ ID NO 8 and an influenza M1 protein. In another embodiment, said VLP comprises the amino acid sequence that codes for any one of SEQ ID NO 2 to SEQ ID NO 8 and a HIV p55 Gag protein. Examples of this constructs are illustrated in FIG. 1.

In another embodiment, said glycoproteins from viruses may comprise: F and/or G protein from RSV, HA and/or NA from influenza virus (including avian or pandemic), S protein from coronavirus, gp160, gp140 and/or gp41 from HIV, or derivatives thereof, gp I to IV, Vp and gE from varicella zoster virus, gE and preM/M from yellow fever virus, Dengue virus (all serotypes) or any flavivirus. Also included are any glycoprotein from a virus that can induce an immune response (cellular and/or humoral) in an animal that can prevent, treat, manage and/or ameliorate an infectious disease in said animal.

Non-limiting examples of bacteria from which said proteins can be expressed in VLPs of the invention are from the following: B. pertussis, Leptospira pomona, S. paratyphi A and B, C. diphtheriae, C. tetani, C. botulinum, C. perfringens, C. feseri and other gas gangrene bacteria, B. anthracis, P. pestis, P. multocida, Neisseria meningitidis, N. gonorrheae, Hemophilus influenzae, Actinomyces (e.g., Norcardia), Acinetobacter, Bacillaceae (e.g., Bacillus anthrasis), Bacteroides (e.g., Bacteroides fragilis), Blastomycosis, Bordetella, Borrelia (e.g., Borrelia burgdorferi), Brucella, Campylobacter, Chlamydia, Coccidioides, Corynebacterium (e.g., Corynebacterium diptheriae), E. coli (e.g., Enterotoxigenic E. coli and Enterohemorrhagic E. coli), Enterobacter (e.g. Enterobacter aerogenes), Enterobacteriaceae (Klebsiella, Salmonella (e.g., Salmonella typhi, Salmonella enteritidis, Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes), Mycoplasma, Mycobacterium (e.g., Mycobacterium leprae and Mycobacterium tuberculosis), Vibrio (e.g., Vibrio cholerae), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Moraxella, Meningiococcus, enterococcus, tularemia (Francisella), bartonella, Pneumococcus and Streptococcus (e.g., Streptococcus pneumoniae and Groups A, B, and C Streptococci), Ureaplasmas. Treponema pollidum, Staphylococcus aureus, Pasteurella haemolytica, Corynebacterium diptheriae toxoid, Bordetella pertusis, Streptococcus pneumoniae, Clostridium tetani toxoid, and Mycobacterium bovis.

Non-limiting examples of parasites from which said glycoproteins can be derived from are from the following: leishmaniasis (Leishmania tropica mexicana, Leishmania tropica, Leishmania major, Leishmania aethiopica, Leishmania braziliensis, Leishmania donovani, Leishmania infantum, Leishmania chagasi), trypanosomiasis (Trypanosoma brucei gambiense, Trypanosoma brucei rhodesiense), toxoplasmosis (Toxoplasma gondii), schistosomiasis (Schistosoma haematobium, Schistosoma japonicum, Schistosoma mansoni, Schistosoma mekongi, Schistosoma intercalatum), malaria (Plasmodium virax, Plasmodium falciparium, Plasmodium malariae and Plasmodium ovale) Amebiasis (Entamoeba histolytica), Babesiosis (Babesiosis micron), Cryptosporidiosis (Cryptosporidium parvum), Dientamoebiasis (Dientamoeba fragilis), Giardiasis (Giardia lamblia), Helminthiasis, Trichomonas (Trichomonas vaginalis), Naegleria, Enterobius vermicularis, Onchocerca, ascariasis, Balantidium coli, Ancylostoma, filariasis, microsporidia, strongyloides, taperwoims, tineas, toxocariasis, trichinosis, trichuriasis, trypanosomiasis, hookworm, isosporiasis, and paragonimiasis.

Non-limiting examples of fungi from which said glycoproteins can be derived are from the following: Absidia (e.g. Absidia corymbifera), Ajellomyces (e.g. Ajellomyces capsulatus, Ajellomyces dermatitidis), Arthroderma (e.g. Arthroderma benhamiae, Arthroderma fulvum, Arthroderma gypseum, Arthroderma incurvatum, Arthroderma otae, Arthroderma vanbreuseghemii), Aspergillus (e.g. Aspergillus fumigatus, Aspergillus niger), Candida (e.g. Candida albicans, Candida albicans var. stellatoidea, Candida dublinensis, Candida glabrata, Candida guilliermondii (Pichia guilliermondii), Candida krusei (Issatschenkia orientalis), Candida parapsilosis, Candida pelliculosa (Pichia anomala), Candida tropicalis), Coccidioides (e.g. Coccidioides immitis), Cryptococcus (e.g. Cryptococcus neoformans (Filobasidiella neoformans), Histoplasma (e.g. Histoplasma capsulatum (Ajellomyces capsulatus), Microsporum (e.g. Microsporum canis (Arthrodermaotae), Microsporum fulvum (Arthroderma fulvum), Microsporum gypseum, Genus Pichia (e.g. Pichia anomala, Pichia guilliermondii), Pneumocystis (e.g. Pneumocystis jirovecii), Cryptosporidium, Malassezia furfur, Paracoccidiodes.

Said glycoproteins may be derived from tumor associated antigens. For example, specific tumor-associated antigens include, but not limited to, Ras p21 protooncogenes, tumor suppressor p53 and HER-2/neu and BCR-abl oncogenes, CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG), Mart 1/Melan A, gp100, gp75, Tyrosinase, TRP1, PSA, PAP, PSMA, and PSM-P1. The above lists are meant to be illustrative and by no means are meant to limit the invention to those particular bacterial, viral, fungal, parasitic or tumor associated proteins.

The invention also encompasses variants of the said glycoproteins (or proteins) expressed on or in the VLPs of the invention (including said chimeras). The variants may contain alterations in the amino acid sequences of the constituent proteins. The telin “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif (Berger); Sambrook et al., Molecular Cloning—A. Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutation of chimeric glycoproteins. Thus, the invention also encompasses using known methods of protein engineering and recombinant DNA technology to improve or alter the characteristics of the glycoproteins expressed on or in the VLPs of the invention. Various types of mutagenesis can be used to produce and/or isolate glycoproteins and/or to further modify/mutate said glycoprotein. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Nucleotide variants can be produced for a variety of reasons, e.g., to optimize codon expression for a particular host (change codons to those preferred by insect cells such as Sf9 cells, see US 20040121465, herein incorporated by reference for all purposes). Additional suitable methods include point mismatch repair, mutagenesis using repair-deficient host strains, restriction-selection and restriction-purification, deletion mutagenesis, mutagenesis by total gene synthesis, double-strand break repair, and the like. In one embodiment, mutagenesis can be guided by known information of the naturally occurring molecule or altered or mutated naturally occurring molecule, e.g., sequence, sequence comparisons, physical properties, crystal structure or the like.

The invention further comprises protein variants which show substantial biological activity, e.g., able to elicit an effective antibody response when expressed on or in VLPs of the invention. Such variants include deletions, insertions, inversions, repeats, and substitutions selected according to general rules known in the art so as have little effect on activity. An example of a mutation is to remove the cleavage site in a protein.

Methods of cloning said glycoproteins are known in the art. For example, the gene encoding a chimeric glycoprotein (or protein) can be chemically synthesized as a synthetic gene or can be isolated by RT-PCR from polyadenylated mRNA extracted from cells which had been infected with a virus or other organism. The resulting gene product can be cloned as a DNA insert into a vector. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids or bacmids.

The invention also comprises nucleotides that encode chimeric glycoproteins. In one embodiment, the invention comprises nucleotides that encode a chimeric glycoprotein comprising an influenza hemagglutinin transmembrane domain. In another embodiment, the invention comprises nucleotides that encode a chimeric glycoprotein comprising an influenza hemagglutinin c-terminal domain. In another embodiment, the invention comprises nucleotides that encode a chimeric glycoprotein comprising an influenza hemagglutinin transmembrane domain and a hemagglutinin c-terminal domain. In another embodiment, said nucleic acid sequence encodes for a signal sequence derived from the gene of an insect. In another embodiment, said signal sequence is from the chitinase gene. In another embodiment, said nucleic acid sequences comprise the sequence that codes for any one of SEQ ID NO 2 to SEQ ID NO 8.

The invention also comprises nucleotides that encode chimeric glycoproteins cloned into an expression vector that can be expressed in a cell that induces the formation of VLPs. An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In one embodiment, said nucleotides encode for a chimeric glycoprotein (e.g. SEQ ID NO 2 to SEQ ID NO 8). In another embodiment, said nucleotides encode for a chimeric glycoprotein and an influenza M1 protein. In another embodiment, said nucleotides encode for a chimeric glycoprotein and a p55 Gag. In another embodiment, said vector comprises nucleotides that encode the influenza p55 Gag protein and at least any one of SEQ ID NO 2 to SEQ ID NO 8. In another embodiment, said vector comprises nucleotides that encode the influenza M1 protein and at least any one of SEQ ID NO 2 to SEQ ID NO 8. In another embodiment, the expression vector is a baculovirus vector.

Baculoviruses are DNA viruses in the family Baculoviridae. These viruses are known to have a narrow host-range that is limited primarily to Lepidopteran species of insects (butterflies and moths). The baculovirus Autographa californica Nuclear Polyhedrosis Virus (AcNPV), which has become the prototype baculovirus, replicates efficiently in susceptible cultured insect cells. AcNPV has a double-stranded closed circular DNA genome of about 130,000 base-pairs and is well characterized with regard to host range, molecular biology, and genetics. Recombinant baculoviruses that express foreign genes are constructed by way of homologous recombination between baculovirus DNA and chimeric plasmids containing the gene sequence of interest. Methods of constructing recombinant baculovirus are well known in the art (see U.S. Pat. No. 5,762,939). Methods of producing VLPs from recombinant baculovirus are described in U.S. 20040121465, herein incorporated by reference in its entirety for all purposes. In one embodiment, the invention comprises a recombinant baculovirus that comprises at least one chimeric glycoprotein of the invention.

In some embodiments, mutations containing alterations that produce silent substitutions, additions, or deletions, but do not alter the properties or activities of the encoded protein or how the proteins are made are contemplated as an embodiment of the invention. Nucleotide variants can be produced for a variety of reasons, e.g., to optimize codon expression for a particular host See U.S. patent publication 2005/0118191, herein incorporated by reference in its entirety for all purposes.

The invention also provides for constructs and/or vectors that comprise chimeric glycoproteins of the invention. The vector may be, for example, a phage, plasmid, viral, or retroviral vector. The constructs and/or vectors that encode said chimeric glycoproteins should be operatively linked to an appropriate promoter, such as the AcMNPV polyhedrin promoter (or other baculovirus), phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome binding site for translation. The coding portion of the transcripts expressed by the constructs will preferably include a translation initiating codon at the beginning and a termination codon appropriately positioned at the end of the polypeptide to be translated.

The expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. Other suitable vectors will be readily apparent to the skilled artisan. In one embodiment, the vector comprises nucleotides that encode a chimeric glycoprotein comprising an influenza hemagglutinin c-terminal domain. In another embodiment, said vector comprises nucleotides that encode a chimeric glycoprotein comprising an influenza hemagglutinin transmembrane domain and a hemagglutinin c-terminal domain. In another embodiment, said nucleic acid sequence encodes for a signal sequence derived from the gene of an insect. In another embodiment, said signal sequence is from the chitinase gene. In another embodiment, said nucleic acid sequences comprise at least any one of the sequences that codes for SEQ ID NO 2 to SEQ ID NO 8. In another embodiment, said vector is a baculovirus that comprises said nucleic acids which code for said chimeric glycoprotein(s).

Next, the recombinant constructs mentioned above can be transfected, infected, or transformed into eukaryotic cells and/or prokaryotic cells and expressed under conditions that allow VLP formation. Thus, the invention provides for host cells that comprise a vector (or vectors) that contain nucleic acids comprising the constructs described above.

Among eukaryotic host cells are yeast, insect, avian, plant, C. elegans (or nematode) and mammalian host cells. Non limiting examples of insect cells are, Spodoptera frugiperda (Sf) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K. lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, and African green monkey cells, CV1 cells, HeLa cells, MDCK cells, Vero and Hep-2 cells. Xenopus laevis oocytes, or other cells of amphibian origin, may also be used. Prokaryotic host cells include bacterial cells, for example, E. coli, B. subtilis, and mycobacteria. In one embodiment, the recombinant constructs mentioned above could be used to transfect, infect, or transform and can express chimeric glycoproteins and/or influenza M1 and/or p55 Gag in eukaryotic cells and/or prokaryotic cells.

Vectors, e.g., vectors comprising polynucleotides the above described constructs, can be transfected into host cells according to methods well known in the art. For example, introducing nucleic acids into eukaryotic cells can be by calcium phosphate co-precipitation, electroporation, microinjection, lipofection, and transfection employing polyamine transfection reagents. In one embodiment, said vector is a recombinant baculovirus. In another embodiment, said recombinant baculovirus is transfected into a eukaryotic cell. In a preferred embodiment, said cell is an insect cell. In another embodiment, said insect cell is a Sf9 cell.

In a specific embodiment, cells derived from the Lepidopteran species Spodoptera frugiperda are infected with recombinant baculovirus comprising chimeric glycoprotein(s) of the invention and expended in cell culture to produce VLPs. Other insect cells that can be infected by baculovirus, such as those from the species Bombix mori, Galleria mellanoma, Trichplusia ni, or Lamanthnia dispar, could also be used as a suitable host cell to produce VLPs. In one embodiment, the invention comprises a host cell comprising said recombinant baculovirus, wherein said baculovirus encodes for chimeric glycoprotein(s) of the invention. In another embodiment, said host cell is a Sf9 host cell.

The invention also provides for methods of producing VLPs, said methods comprising expressing a chimeric glycoproteins and/or influenza M1 or HIV p55 Gag or other non-influenza core or matrix proteins under conditions that allow VLP foimation. Depending on the expression system and host cell selected, the VLPs are produced by growing host cells transformed by an expression vector under conditions whereby the recombinant proteins are expressed and VLPs are formed. The selection of the appropriate growth conditions is within the skill of a person in the art.

This invention also provides for constructs and methods that will increase the efficiency of VLP production. For example, the addition of leader sequences to the constructs described above can improve the efficiency of protein transporting within the cell. For example, a heterologous signal sequence can be fused to any chimeric glycoprotein described above. In one embodiment, the signal sequence can be derived from the gene of an insect cell. In another embodiment, the signal peptide is the chitinase signal sequence, which works efficiently in baculovirus expression systems.

Another method to increase efficiency of VLP production is to codon optimize the nucleotides that encode any chimeric protein described above and/or influenza M1 or HIV p55 Gag or other non-influenza core or matrix proteins for a specific cell type. For examples of codon optimizing nucleic acids for expression in Sf9 cells see U.S. patent publication 2005/0118191, herein incorporated by reference in its entirety for all purposes.

The invention also provides for methods of producing VLPs, said methods comprising expressing a chimeric protein described above under conditions that allow VLP formation. Depending on the expression system and host cell selected, VLPs are produced by growing host cells transformed by an expression vector under conditions whereby the recombinant proteins are expressed and VLPs are formed. In one embodiment, the invention comprises a method of producing a VLP, comprising transfecting vectors encoding at least a chimeric protein described into a suitable host cell and expressing said protein under conditions that allow VLP formation. In another embodiment, said VLP comprises the influenza M1 protein and a chimeric protein described above. In another embodiment, said VLP comprises a non-influenza core or matrix protein and a chimeric protein described above. In another embodiment, said VLP comprises the p55 Gag protein and a chimeric protein described above. In another embodiment, said eukaryotic cell is selected from the group consisting of, yeast, insect, amphibian, avian or mammalian cells. The selection of the appropriate growth conditions is within the skill or a person with skill of one of ordinary skill in the art.

Methods to grow cells engineered to produce VLPs of the invention include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, said bioreactor is a stainless steel chamber. In another embodiment, said bioreactor is a pre-sterilized plastic bag (e.g. Cellbag®, Wave Biotech, Bridgewater, N.J.). In other embodiment, said pre-sterilized plastic bags are about 50 L to 1000 L bags.

The VLPs are then isolated using methods that preserve the integrity thereof, such as by gradient centrifugation, e.g., cesium chloride, sucrose and iodixanol, as well as standard purification techniques including, e.g., ion exchange and gel filtration chromatography. In one embodiment, the invention comprises purified VLPs of the invention. In another embodiment, said VLPs of the invention are at least 50%, 55% 60%, 65%, 70%, 75%, 80%, 85%, 90%, 95%, 96%, 97%, 98%, 99% or greater, free from other molecules (exclusive of solvent) in a mixture. In another embodiment, said VLPs of the invention are substantially free of other viruses, proteins, lipids, and carbohydrates associated with making VLPs of the invention.

The invention also provides for methods of increasing glycoprotein incorporation on the surface of VLPs, comprising expressing a nucleic acid encoding a chimeric glycoprotein in a host cell with a viral core or matrix protein other than influenza M1, wherein said chimeric glycoprotein comprises the transmembrane domain of an influenza hemagglutinin protein. In one embodiment, said chimeric glycoprotein further comprises the carboxyl terminal tail of an influenza hemagglutinin protein. In another embodiment, said nucleic acid is codon optimized for Sf9 cells. In another embodiment, said chimeric glycoprotein further comprises a chitinase signal sequence. In another embodiment, said method further comprises expressing a nucleic acid coding for an influenza M1 protein. In another embodiment, said method further comprises expressing a nucleic acid coding for a HIV gag protein (e.g. p55 Gag). In another embodiment, said viral glycoprotein is selected from the group consisting of a HIV glycoprotein, a RSV glycoprotein, a PIV glycoprotein, an Ebola virus glycoprotein, a herpes virus glycoprotein, a hepatitis virus glycoprotein, an Epstein Barr virus glycoprotein, a corona virus glycoprotein, and combinations thereof. In another embodiment, said glycoprotein is an HIV glycoprotein. In another embodiment said HIV glycoprotein is gp160, gp120, gp41 and/or gp145 or derivatives thereof.

The following is an example of how VLPs of the invention can be made, isolated and purified. Usually VLPs are produced from recombinant cell lines engineered to create VLPs when said cells are grown in cell culture (see above). A person of skill in the art would understand that there are additional methods that can be utilized to make and purify VLPs of the invention, thus the invention is not limited to the method described.

Production of VLPs of the invention can start by seeding Sf9 cells (non-infected) into shaker flasks, allowing the cells to expand and scaling up as the cells grow and multiply (for example from a 125-ml flask to a 50 L Wave bag). The medium used to grow the cell is formulated for the appropriate cell line (preferably serum free media, e.g. insect medium ExCe11-420, JRH). Next, said cells are infected with recombinant baculovirus at the most efficient multiplicity of infection (e.g. from about 1 to about 3 plaque forming units per cell). Once infection has occurred, the influenza M1 protein or HIV p55 Gag and a chimeric protein described above, are expressed from the virus genome, self assemble into VLPs and are secreted from the cells approximately 24 to 72 hours post infection. Usually, infection is most efficient when the cells are in mid-log phase of growth (4-8×10⁶ cells/ml) and are at least about 90% viable.

VLPs of the invention can be harvested approximately 48 to 96 hours post infection, when the levels of VLPs in the cell culture medium are near the maximum but before extensive cell lysis. The Sf9 cell density and viability at the time of harvest can be about 0.5×10⁶ cells/ml to about 1.5×10⁶ cells/ml with at least 20% viability, as shown by dye exclusion assay. Next, the medium is removed and clarified. NaCl can be added to the medium to a concentration of about 0.4 to about 1.0 M, preferably to about 0.5 M, to avoid VLP aggregation. The removal of cell and cellular debris from the cell culture medium containing VLPs of the invention can be accomplished by tangential flow filtration (TFF) with a single use, pre-sterilized hollow fiber 0.5 or 1.00 μm filter cartridge or a similar device.

Next, VLPs in the clarified culture medium can be concentrated by ultrafiltration using a disposable, pre-sterilized 500,000 molecular weight cut off hollow fiber cartridge. The concentrated VLPs can be diafiltrated against 10 volumes pH 7.0 to 8.0 phosphate-buffered saline (PBS) containing 0.5 M NaCl to remove residual medium components.

The concentrated, diafiltered VLPs can be furthered purified on a 20% to 60% discontinuous sucrose gradient in pH 7.2 PBS buffer with 0.5 M NaCl by centrifugation at 6,500×g for 18 hours at about 4° C. to about 10° C. Usually VLPs will form a distinctive visible band between about 30% to about 40% sucrose or at the interface (in a 20% and 60% step gradient) that can be collected from the gradient and stored. This product can be diluted to comprise 200 mM of NaCl in preparation for the next step in the purification process. This product contains VLPs and may contain intact baculovirus particles.

Further purification of VLPs can be achieved by anion exchange chromatography, or 44% isopycnic sucrose cushion centrifugation. In anion exchange chromatography, the sample from the sucrose gradient (see above) is loaded into column containing a medium with an anion (e.g. Matrix Fractogel EMD TMAE) and eluded via a salt gradient (from about 0.2 M to about 1.0 M of NaCl) that can separate the VLP from other contaminates (e.g. baculovirus and DNA/RNA). In the sucrose cushion method, the sample comprising the VLPs is added to a 44% sucrose cushion and centrifuged for about 18 hours at 30,000 g. VLPs form a band at the top of 44% sucrose, while baculovirus precipitates at the bottom and other contaminating proteins stay in the 0% sucrose layer at the top. The VLP peak or band is collected.

The intact baculovirus can be inactivated, if desired. Inactivation can be accomplished by chemical methods, for example, formalin or β-propiolactone (BPL). Removal and/or inactivation of intact baculovirus can also be largely accomplished by using selective precipitation and chromatographic methods known in the art, as exemplified above. Methods of inactivation comprise incubating the sample containing the VLPs in 0.2% of BPL for 3 hours at about 25° C. to about 27° C. The baculovirus can also be inactivated by incubating the sample containing the VLPs at 0.05% BPL at 4° C. for 3 days, then at 37° C. for one hour.

After the inactivation/removal step, the product comprising VLPs can be run through another diafiltration step to remove any reagent from the inactivation step and/or any residual sucrose, and to place the VLPs into the desired buffer (e.g. PBS). The solution comprising VLPs can be sterilized by methods known in the art (e.g. sterile filtration) and stored in the refrigerator or freezer.

The above techniques can be practiced across a variety of scales. For example, T-flasks, shake-flasks, spinner bottles, up to industrial sized bioreactors. The bioreactors can comprise either a stainless steel tank or a pre-sterilized plastic bag (for example, the system sold by Wave Biotech, Bridgewater, N.J.). A person with skill in the art will know what is most desirable for their purposes.

### Vaccine or Immunogenic Formulations and Administration

The pharmaceutical compositions useful herein contain a pharmaceutically acceptable carrier, including any suitable diluent or excipient, which includes any pharmaceutical agent that does not itself induce the production of an immune response harmful to the animal receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in animals, and more particularly in humans. These compositions can be useful as a vaccine and/or immunogenic compositions for inducing a protective immune response in an animal.

Said pharmaceutical formulations of the invention comprise VLPs comprising a chimeric glycoprotein and/or influenza M1 protein and/or influenza p55 Gag protein, or another viral core or matrix protein and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

Generally, VLPs of the invention are administered in an effective amount or quantity sufficient to stimulate an immune response against the glycoprotein of the surface of the VLP. Typically, the dose can be adjusted within this range based on, e.g., age, physical condition, body weight, sex, diet, time of administration, and other clinical factors. The prophylactic vaccine or immunogenic composition is systemically administered, e.g., by subcutaneous or intramuscular injection using a needle and syringe, or a needle-less injection device. Alternatively, the vaccine or immunogenic composition is administered intranasally, either by drops, large particle aerosol (greater than about 10 microns), or spray into the upper respiratory tract.

Thus, the invention also comprises a method of formulating a vaccine or immunogenic composition that induces a protective immune response comprising adding to said formulation an effective dose of a VLP of the invention. In one embodiment, the invention comprises a vaccine or immunogenic composition, wherein said vaccine and/or composition comprises a VLP of the invention. In another embodiment, said vaccine or immunogenic composition further comprises a pharmaceutically acceptable carrier.

While stimulation of the immune system with a single dose is preferred, additional dosages can be administered, by the same or different route, to achieve the desired effect. In neonates and infants, for example, multiple administrations may be required to elicit sufficient levels of immunity. Levels of induced immunity can be monitored, for example, by measuring amounts of neutralizing secretory and serum antibodies, and dosages adjusted or vaccinations repeated as necessary to elicit and maintain desired levels of protection.

Vaccines and/or immunogenic formulations of the invention may also be administered on a dosage schedule, for example, an initial administration of the vaccine composition with subsequent booster administrations.

The dosage of the pharmaceutical formulation can be determined readily by the skilled artisan, for example, by first identifying doses effective to elicit a prophylactic or therapeutic immune response, e.g., by measuring the serum titer of specific immunoglobulins or by measuring the inhibitory ratio of antibodies in serum samples, or urine samples, or mucosal secretions. Said dosages can be determined from animal studies. A non-limiting list of animals used to study efficacy of VLPs of the invention include the guinea pig, Syrian hamster, chinchilla, hedgehog, chicken, rat, mouse, ferret, and primates.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

As also well known in the art, the immunogenicity of a particular composition can be enhanced by the use of non-specific stimulators of the immune response, known as adjuvants.

Adjuvants have been used experimentally to promote a generalized increase in immunity against unknown antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2^{nd} Edition),” herein incorporated by reference in its entirety for all purposes, is envisioned within the scope of this invention. In one embodiment, said vaccine or immunogenic composition of the invention comprises an adjuvant.

Exemplary, adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is contemplated. MF-59, Novasomes®, MHC antigens may also be used.

In one embodiment of the invention, the adjuvant is a paucilamellar lipid vesicle having about two to ten bilayers arranged in the form of substantially spherical shells separated by aqueous layers surrounding a large amorphous central cavity free of lipid bilayers. Paucilamellar lipid vesicles may act to stimulate the immune response several ways, as non-specific stimulators, as carriers for the antigen, as carriers of additional adjuvants, and combinations thereof. Paucilamellar lipid vesicles act as non-specific immune stimulators when, for example, a vaccine is prepared by intermixing the antigen with the preformed vesicles such that the antigen remains extracellular to the vesicles. By encapsulating an antigen within the central cavity of the vesicle, the vesicle acts both as an immune stimulator and a carrier for the antigen. In another embodiment, the vesicles are primarily made of nonphospholipid vesicles. In other embodiment, the vesicles are Novasomes. Novasomes® are paucilamellar nonphospholipid vesicles ranging from about 100 nm to about 500 nm. They comprise Brij 72, cholesterol, oleic acid and squalene. Novasomes have been shown to be an effective adjuvant for influenza antigens (see, U.S. Pat. Nos. 5,629,021, 6,387,373, and 4,911,928, herein incorporated by reference in their entireties for all purposes).

In one aspect, an adjuvant effect is achieved by use of an agent, such as alum, used in about 0.05 to about 0.1% solution in phosphate buffered saline. Alternatively, the VLPs can be made as an admixture with synthetic polymers of sugars (Carbopol®) used as an about 0.25% solution. Some adjuvants, for example, certain organic molecules obtained from bacteria; act on the host rather than on the antigen. An example is muramyl dipeptide (N-acetylmuramyl-L-alanyl-D-isoglutamine [MDP]), a bacterial peptidoglycan. In other embodiments, hemocyanins and hemoerythrins may also be used with VLPs of the invention. The use of hemocyanin from keyhole limpet (KLH) is preferred in certain embodiments, although other molluscan and arthropod hemocyanins and hemoerythrins may be employed.

Another method of inducing an immune response can be accomplished by formulating the VLPs of the invention with “immune stimulators.” These are the body's own chemical messengers (cytokines) to increase the immune system's response. Immune stimulators include, but not limited to, various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the VLPs of the invention or can be administered separately. Either the protein or an expression vector encoding the protein can be administered to produce an immunostimulatory effect.

### Methods of Stimulating an Immune Response

The VLPs of the invention are useful for preparing compositions that stimulate an immune response that confers immunity or substantial immunity to diseases caused by viruses (e.g. HIV), bacteria, fungi, parasites, and/or cancer antigens. Both mucosal and cellular immunity may contribute to immunity against diseases. Antibodies secreted locally in the upper respiratory tract are a major factor in resistance to natural infection. Secretory immunoglobulin A (sIgA) is involved in protection of the upper respiratory tract and serum IgG in protection of the lower respiratory tract. The immune response induced by an infection protects against reinfection with the same virus or an antigenically similar viral strain. Some viruses, like influenza, undergo frequent and unpredictable changes; therefore, after natural infection, the effective period of protection provided by the host's immunity may only be a few years against the new strains of virus circulating in the community.

VLPs of the invention can induce immunity in an animal (e.g. a human) when administered to said animal. The immunity results from an immune response against a VLP of the invention that protects or ameliorates an infection or at least reduces a symptom of an infection in said animal. In some instances, if the said animal is infected, said infection will be asymptomatic. The response may be not a fully protective response. In this case, if said animal is infected with an infectious organism, the animal will experience reduced symptoms or a shorter duration of symptoms compared to a non-immunized animal.

In one embodiment, the invention comprises a method of inducing immunity to a disease or at least one symptom thereof in a subject, comprising administering at least one effective dose of a VLP of the infection. In another embodiment, the invention comprises a method of vaccinating an animal against a disease comprising administering to said animal a protection-inducing amount of a VLP comprising at least one chimeric glycoprotein described above. In another embodiment, the invention comprises a method of vaccinating an animal against HIV comprising administering to said animal a protection-inducing amount of a VLP comprising at least one chimeric glycoprotein described above. In one embodiment, said method comprises administering a VLP comprising p55 Gag proteins. In one embodiment, said method comprises administering a VLP comprising influenza M1 protein.

In another embodiment, the invention comprises a method of inducing a protective antibody response to a disease or at least one symptom thereof in an animal, comprising administering at least one effective dose of a VLP, wherein said VLP comprises the chimeric glycoprotein described above. As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. Antibodies exist as intact immunoglobulins or as a number of well-characterized fragments produced by digestion with various peptidases.

In another embodiment, the invention comprises a method of inducing a protective cellular response to a disease or at least one symptom thereof in a subject, comprising administering at least one effective dose of a VLP, wherein said VLP comprises the chimeric protein described above. Cell-mediated immunity also plays a role in recovery from many viral infections. Cell-mediated immunity also plays a role in recovery from bacterial and parasitic infections.

As mentioned above, the VLPs of the invention prevent or reduce at least one symptom of disease in an animal. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of an infection or additional symptoms, a reduced severity of a symptoms or a suitable assays (e.g. antibody titer and/or T-cell activation assay). The objective assessment comprises both animal and human assessments.

This invention is further illustrated by the following examples that should not be construed as limiting. The contents of all references, patents and published patent applications cited throughout this application, as well as the Figures and the Sequence Listing, are incorporated herein by reference for all purposes.

### EXAMPLES

### Example 1

Construction of chimeric Con-S Env gene. The Con-S ΔCFI gp145 gene, a derivative of the consensus HIV-1 group M ConS env gene which lacks the 120-gp41 cleavage (C) site, the fusion (F) peptide, an immunodominant (I) region in gp41, as well as the CT domain (Liao, H. X., et al. (2006) Virology 353, 268-282). PCR was used to make all the constructs. The PCR products were cloned into vector pBluescript II KS (pBlue) in the polylinker site with BamHI and SalI, and the resulting construct was used to generate chimeric HIV-1 Env mutants.

To construct an influenza HA derived chimeric Con-S ΔCFI Env gene, the HIV-1 signal peptide was replaced with chitinase SP derived from Autographa californica Nuclear Polyhedrosis Virus (AcNPV) chitinase gene. The TM and CT domains of Con-S were replaced with the corresponding C-terminal region of influenza HA that contained putative transmembrane and carboxy terminal sequences derived from influenza A/Fujian/411/02 (H3N2) hemagglutinin. The chimeric gene was codon-optimized for high-level expression in Sf9 cells and synthesized by primer overlapping extension PCR. The resulting PCR fragment was introduced into pFastBac1 transfer vector (Invitrogen) using RsrII and NotI sites within the pFastBac1 polylinker. The identity of all constructs was confirmed by sequence analysis.

Generation of recombinant baculovirus (rBVs). The confirmed chimeric Con-S genes were subcloned into transfer vector pFastBac™ under the polyhedrin promoter. rBVs were generated using the Bac-to-Bac Expression System (Invitrogen) following the manufacturer's instruction. Briefly, pFastBac™I plasmids containing chimeric Con-S ACFI HIV-1 env genes were transformed into DH10Bac competent cells (Invitrogen Life Sciences), white colonies screened in the LB media containing antibiotics kanamycin (50 μg/ml), gentamycin (7 μg/ml), and tetracycline (10 μg/ml) and X-gal and IPTG. After 3 cycles of white colony screening, recombinant Bacmid baculovirus DNAs (rAcNPV) were isolated and transfected into Sf9 insect cells using a Cellfectin reagent (Invitrogen Life Sciences). Transfected culture supernatants were harvested and plaques purified. The expression of chimeric Con-S HIV-1 Env proteins from rBV infected cells was confirmed by western blot.

For the generation of an HIV-1 Gag expressing rBV, codon usage optimized version of the 2002 consensus subtype B gag gene (GenBank accession number EF428978) was synthesized. To generate rBVs of the influenza M1, Gag, their encoding sequences (Deen, K. C., et al. (1986) J. Virol., 57, 422-432, Galarza, J. M., et al. (2005) Viral Immunol., 18, 244-251) were subcloned into transfer vector pFastBac™I under the polyhedrin promoter. These resulting pFastBac™I plasmids were used to generate rBVs following the same procedure as used for the generation of the chimeric Con-S Env rBVs as described above. The protein expression from rBV-infected insect cells was confirmed by western blot.

Cell surface expression assay. Sf9 cells were seeded to 6-well plates at 1×10⁶ cells/well. Recombinant BV infection, expression and isotopic labeling were performed as described (Yamshchikov, G. V., et al. (1995) Virology, 214, 50-58) with modification. In brief, Sf9 cells were infected with rBV at a M.O.I. of 4 PFU/cell for 1 hr at RT. The inoculum was removed and replaced with fresh Sf-900 II SFM medium (Gibco) plus 1% fetal bovine serum (FBS). At 48 hr postinfection, virus-infected cells were placed in methionine and cysteine-free SF-900 II SFM medium for 45 min. Cells were then labeled with 250 μCi/ml of [³⁵S]methionine/cysteine (Amersham) for 4 hr. Biotinylation of cell surface proteins was carried out as described (Yang, C., et al. (1996) Virology, 221, 87-97). The final samples were loaded onto SDS-PAGE. Gels were dried and then used for X-ray film exposure and phosphorImager analysis.

Determination of Env and Gag contents in VLPs. A sandwich ELISA was employed for Env quantitation. Goat anti-HIV-1 gp120 polyclonal antibody was used as a coating antibody and a mixture of monoclonal antibodies, b12 and F425, were used as detection antibodies. HIV-1 SF162 gp120 (NIH AIDS Research and Reference Reagent Program, catalog number: 7363) was used as calibration standard. For Gag quantitation, a commercial HIV-1 p24 kit (Beckman Coulter) was used following the manufacturer's protocol. A sf2 p55 (NIH AIDS Research and Reference Reagent Program, catalog Number: 5109) was used as a calibration standard.

### Example 2

Design of chimeric Env proteins. The following gp145 sequences and derivatives from HIV-1 were cloned into the pFastBac1 (InVitrogen), resulting pFastBac1-based plasmids expressing of HIV gp145 glycoproteins and chimeric constructs.

gp45
SEQ ID NO. 1 -Wild type
Signal peptide shown in bold, HIV Transmembrane underlined

| | | | | |
|---|---|---|---|---|
| MRVRGIQRNC | QHLWRWGTLI | LGMLMICSAA | ENLWVTVYYG | VPVWKEANTT |
| LFCASDAKAY | DTEVHNVWAT | HACVPTDPNP | QEIVLENVTE | NFNMWKNNMV |
| EQMHEDIISL | WDQSLKPCVK | LTPLCVTLNC | TNVNVTNTTN | NTEEKGEIKN |
| CSFNITTEIR | DKKQKVYALF | YRLDVVPIDD | NNNNSSNYRL | INCNTSAITQ |
| ACPKVSFEPI | PIHYCAPAGF | AILKCNDKKF | NGTGPCKNVS | TVQCTHGIKP |
| VVSTQLLLNG | SLAEEEIIIR | SENITNNAKT | IIVQLNESVE | INCTRPNNNT |
| RKSIRIGPGQ | AFYATGDIIG | DIRQAHCNIS | GTKWNKTLQQ | VAKKLREHFN |
| NKTIIFKPSS | GGDLEITTHS | FNCRGEFFYC | NTSGLFNSTW | IGNGTKNNNN |
| TNDTITLPCR | IKQIINMWQG | VGQAMYAPPI | EGKITCKSNI | TGLLLTRDGG |
| NNNTNETEIF | RPGGGDMRDN | WRSELYKYKV | VKIEPLGVAP | TKAKLTVQAR |
| QLLSGIVQQQ | SNLLRAIEAQ | QHLLQLTVWG | IKQLQARVLA | VERYLKDQQL |
| LEIWDNMTWM | EWEREINNYT | DIIYSLIEES | QNQQEKNEQE | LLALDKWASL |

| | | |
|---|---|---|
| WNWFDITNWL | WYIKIFIMIV GGLIGLRIVF | AVLSI |

SEQ ID NO 2
Chitinase signal peptide shown in small case, HIV Transmembrane underlined

| | | | | |
|---|---|---|---|---|
| myplykllnvl | wlvavsnaip | AENLWVTVYY | GPVWKEANT | T |
| LFCASDAKAY | DTEVHNVWAT | HACVPTDPNP | QEIVLENVTE | NFNMWKNNMV |
| EQMHEDIISL | WDQSLKPCVK | LTPLCVTLNC | TNVNVTNTTN | NTEEKGEIKN |
| CSFNITTEIR | DKKQKVYALF | YRLDVVPIDD | NNNNSSNYRL | INCNTSAITQ |
| ACPKVSFEPI | PIHYCAPAGF | AILKCNDKKF | NGTFPCKNVS | TVQCTHGIKP |
| VVSTQLLLNG | SLAEEEIIIR | SENITNNAKT | IIVQLNESVE | INCTRPNNNT |
| RKSIRIGPGQ | AFYATGDIIG | DIRQAHCNIS | GTKWNKTLQQ | VAKKLREHFN |
| NKTIIFKPSS | GGDLEITTHS | FNCRGEFFYC | NTSGLFNSTW | IGNGTKNNNN |
| TNDTITLPCR | IKQIINMWQG | VGQAMYAPPI | EGKITCKSNI | TGLLLTRDGG |
| NNNTNETEIF | RPGGGDMRDN | WRSELYKYKV | VKIEPLGVAP | TKAKLTVQAR |
| QLLSGIVQQQ | SNLLRAIEAQ | QHLLQLTVWG | IKQLQARVLA | VERYLKDQQL |
| LEIWDNMTWM | EWEREINNYT | DIIYSLIEES | QNQQEKNEQE | LLALDKWASL |

| | | |
|---|---|---|
| WNWFDITNWL | WYIKIFIMIV GGLIGLRIVF | AVLSI |

SEQ ID NO 3
Codon-optimized for expression in Sf9 cells, chitinase signal peptide shown
in small case, HIV Transmembrane underlined

| | | | | |
|---|---|---|---|---|
| Mplykllnvl | wlvavsnaip | AENLWVTVYY | GVPVWKEANT | T |
| LFCASDAKAY | DTEVHNVWAT | HACVPTDPNP | QEIVLENVTE | NFNMWKNNMV |
| EQMHEDIISL | WDQSLKPCVK | LTPLCVTLNC | TNVNVTNTTN | NTEEKGEIKN |
| CSFNITTEIR | DKKQKVYALF | YRLDVVPIDD | NNNNSSNYRL | INCNTSAITQ |
| ACPKVSFEPI | PIHYCAPAGF | AILKCNDKKF | NGTGPCKNVS | TVQCTHGIKP |
| VVSTQLLLNG | SLAEEEIIIR | SENITNNAKT | IIVQLNESVE | INCTRPNNNT |
| RKSIRIGPGQ | AFYATGDIIG | DIRQAHCNIS | GTKWNKTLQQ | VAKKLREHFN |
| NKTIIFKPSS | GGDLEITTHS | FNCRGEFFYC | NTSGLFNSTW | IGNGTKNNNN |
| TNDTITLPCR | IKQIINMWQG | VGQAMYAPPI | EGKITCKSNI | TGLLLTRDGG |
| NNNTNETEIF | RPGGGDMRDN | WRSELYKYKV | VKIEPLGVAP | TKAKLTVQAR |
| QLLSGIVQQQ | SNLLRAIEAQ | QHLLQLTVWG | IKQLQARVLA | VERYLKDQQL |
| LEIWDNMTWM | EWEREINNYT | DIIYSLIEES | QNQQEKNEQE | LLALDKWASL |

| | | |
|---|---|---|
| WNWFDITNWL | WYIKIFIMIV GGLIGLRIVF | AVLSI |

SEQ ID NO 4
chitinase signal peptide shown in small case, HIV Transmembrane underlined
(HA c-terminus shown shaded)

| | | | | |
|---|---|---|---|---|
| Mplykllnvl | wlvavsnaip | AENLWVTVYY | GVPVWKEANT | T |
| LFCASDAKAY | DTEVHNVWAT | HACVPTDPNP | QEIVLENVTE | NFNMWKNNMV |
| EQMHEDIISL | WDQSLKPCVK | LTPLCVTLNC | TNVNVTNTTN | NTEEKGEIKN |
| CSFNITTEIR | DKKQKVYALF | YRLDVVPIDD | NNNNSSNYRL | INCNTSAITQ |
| ACPKVSFEPI | PIHYCAPAGF | AILKCNDKKF | NGTGPCKNVS | TVQCTHGIKP |
| VVSTQLLLNG | SLAEEEIIIR | SENITNNAKT | IIVQLNESVE | INCTRPNNNT |
| RKSIRIGPGQ | AFYATGDIIG | DIRQAHCNIS | GTKWNKTLQQ | VAKKLREHFN |
| NKTIIFKPSS | GGDLEITTHS | FNCRGEFFYC | NTSGLFNSTW | IGNGTKNNNN |
| TNDTITLPCR | IKQIINMWQG | VGQAMYAPPI | EGKITCKSNI | TGLLLTRDGG |
| NNNTNETEIF | RPGGGDMRDN | WRSELYKYKV | VKIEPLGVAP | TKAKLTVQAR |
| QLLSGIVQQQ | SNLLRAIEAQ | QHLLQLTVWG | IKQLQARVLA | VERYLKDQQL |
| LEIWDNMTWM | EWEREINNYT | DIIYSLIEES | QNQQEKNEQE | LLALDKWASL |

| | |
|---|---|
| WNWFDITNWL | WYIKIFIMIV GGLIGLR WACQKGNIRCNICI |

SEQ ID NO 5
chitinase signal peptide shown in small case, HA tm domain underlined and
shaded, HA c-terminus shaded
ChiSP_ConSDeltaCFItm(−)_HAtm(+)

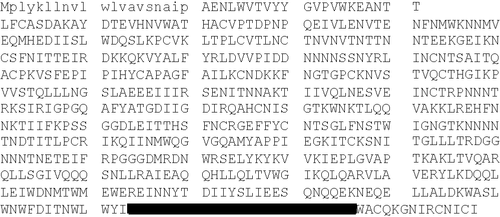

SEQ ID NO 6
chitinase signal peptide shown in small case, HA tm domain underlined and
shaded, HA c-terminus shaded)
ChiSP_ConSDeltaCFItm(−)_HAtm(+)[+1]

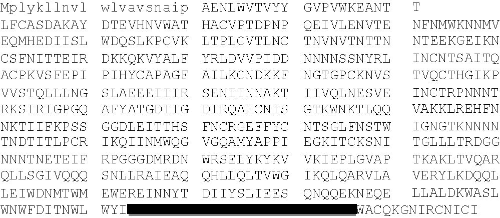

SEQ ID NO 7
chitinase signal peptide shown in small case, HA tm domain underlined and
shaded, HA c-terminus shaded)
ChiSP_ConSDeltaCFItm(−)_HAtm(+)[+2]

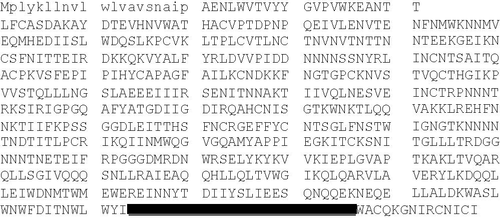

SEQ ID NO 8
ChitinaseSP_HIV
(with ConBenv C-terminus underlined)

| Mplykllnvl | wlvavsnaip | AENLWTVYY | GVPVWKEANT | T |
|---|---|---|---|---|
| LFCASDAKAY | DTEVHNVWAT | HACVPTDPNP | QEIVLENVTE | NFNMWKNNMV |
| EQMHEDIISL | WDQSLKPCVK | LTPLCVTLNC | TNVNVTNTTN | NTEEKGEIKN |
| CSFNITTEIR | DKKQKVYALF | YRLDVVPIDO | NNNNSSNYRL | INCNTSAITQ |
| ACPKVSFEPI | PIHYCAPAGF | AILKCNDKKF | NGTGPCKNVS | TVQCTHGIKP |
| VVSTQLLLNG | SLAEEEIIIR | SENITNNAKT | IIVQLNESVE | INCTRPNNNT |
| RKSIRIGPGQ | AFYATGDIIG | DIRQAHCNIS | GTKWNKTLQQ | VAKKLREHFN |
| NKTIIFKPSS | GGDLEITTHS | FNCRGEFFYC | NTSGLFNSTW | IGNGTKNNNN |
| TNDTITLPCR | IKQIINMWQG | VGQAMYAPPI | EGKITCKSNI | TGLLLTRDGG |
| NNNTNETEIF | RPGGGDMRDN | WRSELYKYKV | VKIEPLGVAP | TKAKLTVQAR |
| QLLSGIVQQQ | SNLLRAIEAQ | QHLLQLTVWG | IKQLQARVLA | VERYLKDQQL |
| LEIWDNMTWM | EWEREINNYT | DIIYSLIEES | QNQQEKNEQE | LLALDKWASL |
| WNWFDITNWL | WYIKIFIMIV GGLIGLR IVFAVLSIVN RVRQGYSPLS | | | |
| FQTRLPAPRG | PDRPEGIEEE | GGERDRDRSG | RLVDGFLALI | WDDLRSLCLF |
| SYHRLRDLLL | IVTRIVELLG | RRGWEVLKYW | WNLLQYWSQE | LKNSAVSLLN |
| ATAIAVAEGT | DRVIEVVQRA | CRAILHIPRR | IRQGLERALL* | |

### Example 3

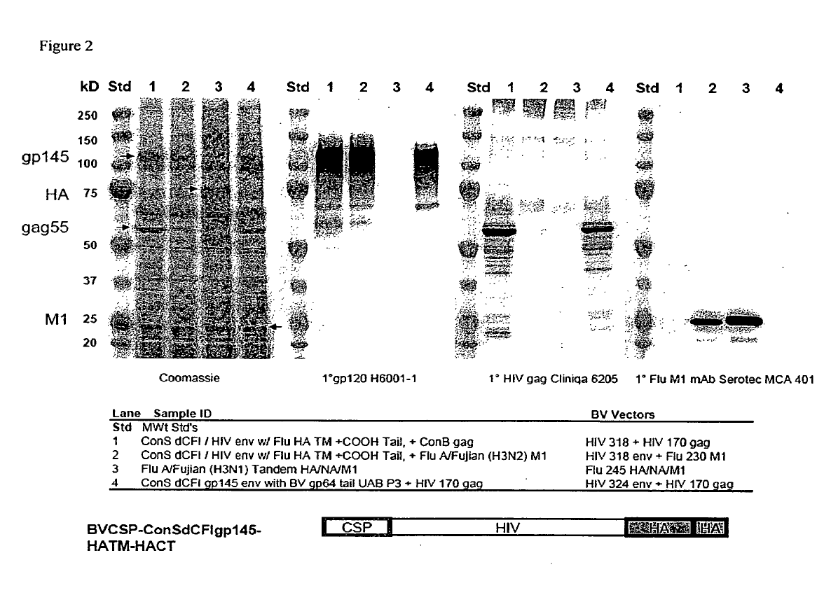

Following production of recombinant baculovirus, Spodoptera frugiperda cells (Sf9) were infected with one or a mixture of recombinant baculovirus (listed on FIG. 1), infected cells were removed by low speed centrifugation from the culture media at about 72 hours post infection. VLP particles recovered from the media using centrifugation at 100,000×g for 1 hour. The VLPs were analyzed (FIG. 2) using SDS-PAGE (left panel) and western blot with anti HIV-1 gp120 (second panel), anti HIV-1 p55 core (third panel), and anti-influenza M1 core (right panel). Lanes 1 and 2 are the HIV-1 env with an influenza transmembrane and C-terminus secreted from Sf9 cells particles when co-expressed with the HIV-1 capsid (p55 core; lane 1) or influenza capsid (M1 matrix; lane 2). Lane 3 is the VLPs with full length influenza A/Fujian HA and M co-expressed in Sf9 cells; the arrow points to recombinant hemagglutinin. Lane 4 is another modified HIV-1 envelope which comprises the transmembrane region and C-terminus of analogous sequences from the baculovirus gp64 envelope gene. The env and gag proteins present in VLPs were also detected with the appropriate anti-HIV-1 antiserum in the second and third panels, respectively. Using this type of hybrid structure also looks to increase expression of HIV-1 envelope. These data show that there is expression of chimeric gp145 proteins on the surface of VLPs.

### Example 4

### Comparison of HIV Chimeric HA Env with Wild-Type

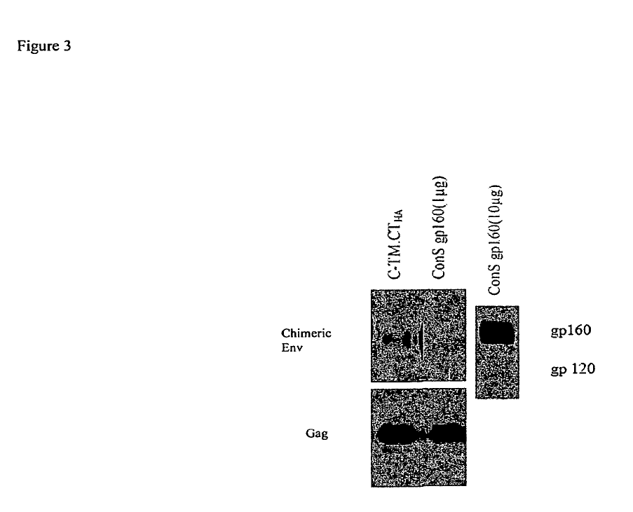

Env incorporation into VLPs with constructs having TM-CT domains derived from influenza virus hemagglutinin (HA) were analyzed. As depicted in FIG. 1, these constructs were generated to have chitinase SP and HA TM-CT (C-TM.CT_{HA}). FIG. 3 depicts a western blot comparing the chimeric and wildtype expression of HIV Env protein. When comparing the C-TM.CTHA lane with ConS gp160(1 ug) lanes, these data demonstrate that the chimeric constructs were found to be incorporated into VLPs at high levels, when compared to the full-length Con-S gp160. The Con-S gp160 VLP did not show detectable Env unless a ten-fold higher quantity of VLPs was loaded for western blot analysis, as shown in the right panel. Data in FIG. 3 show that the Gag/Env molar ratios of HA derived chimeric Env VLPs is 10.8. The ratio for full-length Con-S gp160 VLP was 55.7, demonstrating that heterologous TM and CT domains have dramatic effects by enhancing the levels of incorporation of Env into VLPs. Thus, incorporation of this chimeric Env into VLPs is increase when compared to wildtype.

All references, publications, patents and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of increasing the incorporation of a viral glycoprotein on the surface of a virus-like particle (VLP), comprising expressing a nucleic acid encoding a chimeric glycoprotein in a host cell with a non-influenza viral core, wherein said chimeric glycoprotein comprises the transmembrane domain of an influenza hemagglutinin protein and at least a portion of a viral protein derived from a non-influenza source.

2. (canceled)

3. The method of claim 1, wherein said viral protein is selected from the group consisting of a HIV glycoprotein, a RSV glycoprotein, a PIV glycoprotein, an ebola virus glycoprotein, a herpes virus glycoprotein, a hepatitis virus glycoprotein, an Epstein Barr virus glycoprotein, and a corona virus glycoprotein.

4. (canceled)

5. The method of claim 3, wherein said HIV glycoprotein is gp160 or derivatives thereof.

6. The method of claim 3, wherein said HIV glycoprotein is gp120 or derivatives thereof.

7. The method of claim 3, wherein said HIV glycoprotein is gp145 or derivatives thereof.

8. The method of claim 1, wherein said chimeric glycoprotein further comprises the carboxyl terminal tail of an influenza hemagglutinin protein.

9. The method of claim 1, wherein said nucleic acid encoding said chimeric glycoprotein is expressed from a baculovirus expression system.

10. The method of claim 1, wherein said host cell is an insect cell.

11. The method of claim 1, wherein said non-influenza viral core is HIV p55 Gag.

12. A virus like particle (VLP) comprising at least one chimeric glycoprotein on the surface of the VLP and a non-influenza viral core, wherein said chimeric glycoprotein comprises the transmembrane domain of an influenza hemagglutinin protein and at least a portion of a viral protein derived from a non-influenza source.

13. (canceled)

14. The VLP of claim 12, wherein said chimeric glycoprotein further comprises the carboxyl terminal tail of an influenza hemagglutinin protein.

15. (canceled)

16. The VLP of claim 12, wherein said viral protein is selected from the group consisting of a HIV glycoprotein, a RSV glycoprotein, a PIV glycoprotein, an ebola virus glycoprotein, a herpes virus glycoprotein, a hepatitis virus glycoprotein, an Epstein Barr virus glycoprotein, and a corona virus glycoprotein.

17. (canceled)

18. The VLP of claim 16, wherein said HIV glycoprotein is gp160 or derivatives thereof.

19. The VLP of claim 16, wherein said HIV glycoprotein is gp160 or derivatives thereof.

20. The VLP of claim 16, wherein said HIV glycoprotein is gp160 or derivatives thereof.

21. The VLP of claim 12, wherein said non-influenza viral core is HIV p55 Gag.

22. An immunogenic composition, comprising the VLP of claim 12.

23-25. (canceled)

26. A vaccine comprising the VLP of claim 12.

27-28. (canceled)

29. A method of inducing protective immunity in an animal, comprising administering to said animal a VLP comprising at least one chimeric glycoprotein on the surface of the VLP and a non-influenza viral core, wherein said chimeric glycoprotein comprises the transmembrane domain of an influenza hemagglutinin protein and at least a portion of a viral protein derived from a non-influenza source.

30. (canceled)

31. The method of claim 29, wherein said viral protein is selected from the group consisting of a HIV glycoprotein, a RSV glycoprotein, a PIV glycoprotein, an ebola virus glycoprotein, a herpes virus glycoprotein, a hepatitis virus glycoprotein, an Epstein Barr virus glycoprotein, and a corona virus glycoprotein.

32-37. (canceled)
